(19) United States
(12) Patent Application Publication
(10) Pub. No.: US 2004/0052745 A1
(43) Pub. Date: Mar. 18, 2004

(54) Long-lasting make-up kit and method

(76) Inventors: Pascale Bernard, Sucy-en-Brie (FR); Anke Hadasch, Paris (FR); Beatrice Toumi, Verrieres-le-Buisson (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/332,774

(22) PCT Filed: Jul. 10, 2001
(86) PCT No.: PCT/FR01/02222

(30) Foreign Application Priority Data
Jul. 13, 2000 (FR) 0009216

(51) Int. Cl. A61K 7/06; A61K 7/11

(52) U.S. Cl. 424/70.11

(57) ABSTRACT

The invention concerns a kit for care or make-up comprising at least two cosmetic compositions A and B. Composition A comprises at least an adhesive material. Composition B is a standard cosmetic composition comprising a cosmetically acceptable support as defined for composition A.

The present invention relates to a makeup kit and a process for improving the staying power and/or transfer resistance of a composition, in particular a makeup composition, such as, for example, foundations, powders, lipsticks, mascaras and nail varnishes.

Cosmetic compositions, especially makeup compositions such as lipsticks, foundations, body makeup products, concealer products, eye shadows or powders, generally comprise fatty substances such as oils and/or waxes, and a particulate phase generally composed of fillers and pigments. They may thus be in the form of an anhydrous gel, in the form of a stick or tube or in the form of a soft paste, such as, for example, certain foundations, eye shadows or lipsticks. They may also be in the form of a powder, which may be, for example, free, compacted or pressed. Makeup compositions may also comprise water or a hydrophilic phase, and may then especially be in the form of an oil-in-water emulsion, a water-in-oil emulsion, a multiple emulsion, especially when it is a foundation, a tinted cream, a care cream or an antisun product. Nail varnishes are generally in the form of a solution of an organic solvent.

These compositions do not always show good staying power when they are applied to the skin, mucous membranes or semimucous membranes. Specifically, it has been found that certain compositions have a tendency to travel within the wrinkles and/or fine lines of the skin, in the case of foundations; in the fine lines around the lips, in the case of lipsticks; in the folds of the eyelids, in the case of eye shadows. The appearance of lines in the makeup generated by the movements of the eyelids has also been found, especially in the case of eye shadows. Similarly, the original color is liable to change over time. In the case of nail varnishes, the product is liable to crack, flake or not withstand friction.

Moreover, certain makeup products may have the drawback of transferring. This means that the composition is liable to become at least partially deposited onto certain supports with which it comes into contact, such as, for example, a glass, an item of clothing or the skin.

On becoming deposited, said composition leaves a mark on said support. This therefore results in mediocre persistence of the composition on the skin or mucous membranes, as a result of which it is necessary to renew its application regularly.

Moreover, the appearance of unacceptable traces on certain items of clothing, and especially on blouse collars, may put certain women off using this type of makeup.

These phenomena all result in an unsightly effect that it would obviously be desirable to avoid.

There is thus still a need for products, especially makeup products, that have good staying power while having good cosmetic properties, especially properties of sliding on application, and also softness, moisturization and comfort qualities after applying the makeup.

Now, the Applicant has found, surprisingly, that by combining in a specific manner two cosmetic compositions, each of these two compositions being known per se, it is possible to produce a makeup product that has very good staying power.

Makeup products are thus produced, which not only withstand rubbing, but also have good cosmetic properties, in particular in terms of comfort and moisturization.

The present invention relates to a makeup or care kit comprising at least two cosmetic compositions A and B, characterized in that:

composition A comprises at least one adhesive material that satisfies the following conditions:

G′(2 Hz, 35° C.)≧10³ Pa, and

G′(35° C.)≦10⁸ Pa, preferably G′(35° C.)≦10⁷ Pa,

G′(2×10⁻² Hz, 35° C.)≦3×10⁵ Pa, and

in which:

G′(2 Hz, 35° C.) is the elastic shear modulus of said adhesive material, measured at a frequency of 2 Hz and at a temperature of 35° C.,

G′(35° C.) is the elastic shear modulus of said adhesive material, measured at a temperature of 35° C., for any frequency between 2×10⁻² and 2 Hz,

G′(2×10⁻² Hz, 35° C.) is the elastic shear modulus of said adhesive material, measured at a frequency of 2×10⁻² Hz and at a temperature of 35° C.

The present invention also relates to a process for improving the staying-power and transfer-resistance properties of a cosmetic composition A or B, which consists in applying to the skin and/or mucous membranes and/or semimucous membranes and/or integuments, successively and in any order, the two compositions A and B, at least one of the two compositions A and B comprising at least one adhesive material that satisfies the following conditions:

G′(2 Hz, 35° C.)≧10³ Pa, and

G′(35° C.)≦10⁸ Pa, preferably G′(35° C.)≦10⁷ Pa,

G′(2×10⁻² Hz, 35° C.)≦3×10⁵ Pa, and

in which:

G′(2 Hz, 35° C.) is the elastic shear modulus of said adhesive material, measured at a frequency of 2 Hz and at a temperature of 35° C.,

G′(35° C.) is the elastic shear modulus of said adhesive material, measured at a temperature of 35° C., for any frequency between 2×10⁻² and 2 Hz,

G′(2×10⁻² Hz, 35° C.) is the elastic shear modulus of said adhesive material, measured at a frequency of 2×10⁻² Hz and at a temperature of 35° C.

The makeup or care product obtained on the skin or the integuments according to the above process has the advantage of having excellent staying power and of showing very little transfer or none at all, while conserving its comfort and moisturizing properties. When the composition according to the invention is a nail varnish, it does not flake, chip or crack and it withstands rubbing.

When the compositions according to the invention are applied to the skin, they make it possible, inter alia, to obtain makeup products that have relatively little sticky texture, which remain comfortable to wear throughout the day. Furthermore, their cosmetic properties are very advantageous: they afford considerable final softness and a unifying and comfortable makeup result.

The kit according to the invention especially finds a particularly advantageous use in the field of caring for and/or making up the skin, mucous membranes, semimucous membranes and integuments. The term “mucous membrane” especially means the inner part of the lower eyelid; the term “semimucous membrane” more particularly means the lips of the face; the term “integument” means the eyelashes, eyebrows, hair and nails. Thus, the invention finds a most particular application in the field of care and/or makeup products for the lips, for the face and for the skin, such as foundations, concealer products, eye shadows, powders, body makeup products, lipsticks, self-tanning products or antisun products, mascaras and nail varnishes.

Other characteristics, aspects and advantages of the present invention will become apparent on reading the detailed description that follows.

Composition A according to the invention comprises at least one adhesive material that satisfies the following conditions:

G′(2 Hz, 35° C.)≧10³ Pa, and

G′(35° C.)≦10⁸ Pa, preferably G′(35° C.)≦10⁷ Pa,

G′(2×10⁻² Hz, 35° C.)≦3×10⁵ Pa, and

in which:

G′(2 Hz, 35° C.) is the elastic shear modulus of said adhesive material, measured at a frequency of 2 Hz and at a temperature of 35° C.,

G′(35° C.) is the elastic shear modulus of said adhesive material, measured at a temperature of 35° C., for any frequency between 2×10⁻² and 2 Hz,

G′(2×10⁻² Hz, 35° C.) is the elastic shear modulus of said adhesive material, measured at a frequency of 2×10⁻² Hz and at a temperature of 35° C.

For the purposes of the present invention, the term “material” means a polymer or a polymer system that may comprise one or more polymers of different nature. This adhesive material must have a certain amount of bonding power defined by its viscoelastic properties.

The viscoelastic properties of a material are conventionally defined by two characteristic values, which are as follows:

the elastic modulus, which represents the elastic behavior of the material for a given frequency and which is conventionally written as G′,

the viscous modulus, which represents the viscous behavior of the material for a given frequency, and which is conventionally written as G″.

These magnitudes are especially defined in the “Handbook of Pressure Sensitive Adhesive Technology” 3rd Edition, D. Satas, Chapter 9, pp. 155 to 157.

The adhesive materials that may be used according to the present invention have viscoelastic properties that are measured at a reference temperature of 35° C. and in a certain frequency range. In particular, the elastic modulus of the material is measured at three different frequencies:

at low frequency, i.e. at 2×10⁻² Hz,

at an intermediate frequency, i.e. at 0.2 Hz,

at high frequency, i.e. at 2 Hz,

and the viscous modulus at a frequency of 0.2 Hz.

These measurements make it possible to evaluate the change in the bonding power of the adhesive material over time.

These viscoelastic properties are measured during dynamic tests under sinusoidal stresses of low amplitude (small deformations) performed at 35° C. over a frequency range of from 2×10⁻² to 20 Hz on a “Haake RS50” rheometer under a torsional/shear stress, for example in cone-plate geometry (for example with a cone angle of 1° ).

In one preferred form of the invention, the adhesive material also satisfies the following condition:

G″/G′(0.2 Hz, 35° C.)≧0.35,

in which

G″(0.2 Hz, 35° C.) is the viscous shear modulus of said adhesive material, measured at a frequency of 0.2 Hz and at a temperature of 35° C.,

G′(0.2 Hz, 35° C.) is the elastic shear modulus of said adhesive material, measured at a frequency of 0.2 Hz and at a temperature of 35° C.

In one preferred form of the invention, this gives:

G′(2 Hz, 35° C.)≧5×10³ Pa, and better still G′(2 Hz, 35° C.)≧10⁴ Pa.

In another preferred form of the invention, this gives:

G′(2×10⁻²Hz, 35° C.)≦5×10⁴ Pa.

Preferably also, the adhesive materials according to the invention satisfy the following four conditions:

G′(2 Hz, 35° C.)≧10⁴ Pa, and

G′(35° C.)≦10⁸ Pa, preferably G′(35° C.)≦10⁷ Pa,

G′(2×10⁻²Hz, 35° C.)≦5×10⁴ Pa, and

G″/G′(0.2 Hz, 35° C.)≧0.35.

The adhesive materials according to the invention may be chosen from adhesives of “pressure-sensitive adhesive” type for example, such as those mentioned in the “Handbook of Pressure Sensitive Adhesive Technology” 3rd Edition, D. Satas.

The proadhesive materials according to the invention are preferably adhesive polymers chosen from block and random copolymers comprising at least one monomer or a combination of monomers whose resultant polymer has a glass transition temperature of less than room temperature (25° C.), these monomers or monomer combinations possibly being chosen from butadiene, ethylene, propylene, isoprene, isobutylene and a silicone, and blends thereof. Examples of such materials are block polymers of the styrene-butadiene-styrene, styrene-(ethylene-butylene)-styrene or styrene-isoprene-styrene type, for instance those sold under the trade name “Kraton” from Shell Chemical Co. or “Vector” from Exxon.

The proadhesive materials according to the invention are preferably adhesive polymers chosen from:

polyurethanes,

acrylic polymers,

silicones,

butyl rubbers, especially from polyisobutylenes,

ethylene/vinyl acetate polymers,

polyamides optionally modified with fatty chains,

natural rubbers,

and blends thereof.

In one particularly advantageous form of the invention, the adhesive materials are chosen from polyisobutylenes with a relative molar mass Mv of greater than or equal to 10,000 and less than or equal to 15,000. More preferably, this relative molar mass is greater than or equal to 18,000 and less than or equal to 150,000.

This relative molar mass Mv may be evaluated by its viscosimetric mean, calculated according to the formula J₀=3.06×10⁻² Mv^{0.65}, in which J₀ is the Staudinger index (in cm³/g). This index is calculated from the flow time of a solution with a polyisobutylene concentration of C=0.01 g/cm³ in isooctane, through a capillary I of an Ubbelhode viscometer at 20° C. according to ISO standard 1628.

The relative molar mass Mv may also be evaluated by GPC (gel permeation chromatography) according to the following protocol: 200 μl of a 0.5% solution of polymer (adhesive material) are injected by means of a Waters 6000 A pump, the eluent being a 100% THF solution, flow rate 1 ml/min, at room temperature, through a set of 8 columns: μstyragel 500Å+10⁴Å+2×10³Å+styragel HR0.5 +2×HR1+HR5E (300×7.8 mm). The detection is performed on a Waters 410 refractometer and on a Waters 490 UV detector at a wavelength of 254 nm.

As commercial products that are particularly suitable for the present invention, mention may be made of the polyisobutylenes with respective relative molar masses Mv of 40,000, 55,000 and 85,000 sold under the respective trade names “Oppanol B 10”, “Oppanol B 12” and “Oppanol B 15” by the company BASF, and blends thereof.

The adhesive materials are preferably present in the composition A according to the invention in a content ranging from 0.1% to 99%, preferably from 0.1% to 30% and more preferably from 0.1% to 10% by weight, relative to the total weight of the composition.

Besides the adhesive material defined above, the composition A according to the invention may comprise any cosmetically acceptable support. The expression “cosmetically acceptable support” means a medium that is compatible with any keratin material such as the skin, the nails, the hair, the eyelashes and eyebrows, the mucous membranes and semimucous membranes and any other area of body or facial skin, and also any substitute for these supports, for instance false nails, false eyelashes and hairpieces.

This support may comprise any cosmetically acceptable water-soluble, water-dispersible, liposoluble or lipodispersible compound conventionally used in cosmetics. This support may also comprise any compound which is soluble in a volatile organic solvent, particularly when the composition A according to the invention is a nail varnish. Thus, the support may be in the form of a pulverulent composition, of an anhydrous fatty phase (gel or solution), in the form of an aqueous phase (gel or solution), in the form of a dispersion, of an O/W or W/O emulsion, or of a multiple emulsion, optionally stabilized with one or more organized systems.

For the purposes of the present invention, the expression “organized systems” means inverse micelles or “lyotropic liquid crystal” structures which are formed at room temperature by mixing together several surfactants or mixing together surfactants and polar solvents or mixing together several polar solvents, the polar solvents being chosen, for example, from water, glycerol, panthenol, propylene glycol and butylene glycol, and/or mixtures thereof. The liquid crystal state is a state that is intermediate between the solid state and the liquid state. It is often referred to as the mesomorphic state. These organized systems are thermodynamically stable.

The composition A may thus be a makeup composition. In this case, it may be in the form of a makeup product for the face or the skin, in particular for the body, a foundation, a concealer, a mascara, an eyeliner, a blusher, a face powder, a rouge, an eyeshadow, or a lip makeup product, for instance a lipstick, or alternatively a nail varnish.

Composition A may also be in the form of a dermatological or skincare composition, a makeup base composition or in the form of an antisun composition. In this case, it may optionally comprise cosmetic or dermatological active agents. It may also be used as a care base for the skin or the lips (lip balms, for protecting the lips against the cold and/or sunlight and/or the wind).

Depending on its nature, the adhesive material according to the invention may be present in the support of the composition A in dissolved form or in dispersed form, in an aqueous phase or in an anhydrous phase. The adhesive material according to the invention may thus be in the form of an aqueous dispersion of particles or in the form of an oily dispersion of particles.

The support of the composition A according to the invention may comprise a fatty phase. This fatty phase can preferably comprise at least one cosmetically or physiologically acceptable oil chosen especially from carbon-based, hydrocarbon-based and/or silicone-based oils of mineral, animal, plant or synthetic origin, alone and mixtures thereof, provided that they are compatible with the intended use.

The fatty phase may thus comprise at least one oil chosen from the polyisobutylenes with a relative molar mass Mv of less than or equal to 10,000, for instance the polyisobutylenes of molar mass 455 to 2000 sold under the trade name “Napvis” by the company BP Chemicals or the polyisobutylenes sold under the name “Parleam” by the Ets B. Rossow et Cie.

Mention may also be made of hydrocarbon-based oils such as liquid paraffin or liquid petroleum jelly, mink oil, turtle oil, soybean oil, perhydrosqualene, sweet almond oil, beauty-leaf oil, palm oil, grapeseed oil, sesame seed oil, corn oil, parleam oil, arara oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleyl alcohol or linolenyl alcohol, isostearyl alcohol or octyldodecanol; silicone oils such as polydimethylsiloxanes (PDMS), which are optionally phenylated such as phenyltrimethicones, or optionally substituted with aliphatic and/or aromatic groups, or with functional groups such as hydroxyl, thiol and/or amine groups; polysiloxanes modified with fatty acids, with fatty alcohols or with polyoxyalkylenes, and mixtures thereof.

Advantageously, at least one oil that is volatile at room temperature may be used. The term “volatile oil” means an oil that is capable of evaporating from the skin at room temperature in less than one hour. Preferably, the volatile oil has a viscosity ranging from 0.5 to 25 centistokes at 25° C. After evaporation of these oils, a nonsticky particle deposit is obtained on the skin or mucous membranes. Preferably, oils whose flash point is high enough to allow these oils to be used in the formulation are used. These volatile oils also facilitate the application of the composition to the skin.

These oils may be hydrocarbon-based oils or silicone oils optionally comprising alkyl or alkoxy groups at the end of a silicone chain or pendent.

As volatile silicone oils that may be used in the invention, mention may be made of linear or cyclic silicones containing from 2 to 7 silicon atoms, these silicones optionally comprising alkyl or alkoxy groups containing from 1 to 10 carbon atoms. Mention may thus be made especially of octamethylcyclotetrasiloxane, decamethylcyclopentasiloxane, hexadecamethylcyclohexasiloxane, heptamethylhexyltrisiloxane and heptamethyloctyltrisiloxane, and/or mixtures thereof.

Volatile hydrocarbon-based oils that may be mentioned include C₈-C₁₆ isoparaffins such as isododecane, isodecane, heptane and isohexadecane, and/or mixtures thereof.

These volatile oils may be present in the composition A according to the invention in a content ranging from 0.1% to 99% by weight, preferably from 0.1% to 60% and more preferably from 0.1% to 40%, relative to the total weight of the composition. Preferably, the weight ratio of volatile oil to the adhesive material according to the invention ranges from 1 to 20, more preferably from 1 to 10 and better still from 2 to 4.

The fatty phase may also comprise at least one wax, at least one gum and/or at least one pasty fatty substance, of plant, animal, mineral or synthetic origin, or even silicone-based, and mixtures thereof.

Among the waxes that are solid at room temperature, which may be present in the composition according to the invention, mention may be made of hydrocarbon-based waxes such as beeswax, carnauba wax, candelilla wax, ouricurry wax, Japan wax, cork fiber wax or sugar cane wax, paraffin wax, lignite wax, microcrystalline waxes, lanolin wax, montan wax, ozokerites, polyethylene waxes, the waxes obtained by Fischer-Tropsch synthesis, hydrogenated oils, fatty esters and glycerides that are solid at 25° C. Silicone waxes may also be used, among which mention may be made of alkyl, alkoxy and/or esters of polymethylsiloxane. The waxes may be in the form of stable dispersions of colloidal wax particles as may be prepared according to known methods, such as those of “Microemulsions Theory and Practice”, L. M. Prince Ed., Academic Press (1977), pages 21-32. Mention may be made of jojoba oil as a wax that is liquid at room temperature.

The waxes may be present in a proportion of from 0.1% to 30% by weight relative to the total weight of the composition.

The pasty fatty compounds may be defined by means of at least one of the following physicochemical properties:

a viscosity from 0.1 to 40 Pa.s (1 to 400 poises) and preferably 0.5 to 25 Pa.s, measured at 40° C. with a Contraves TV rotary viscometer equipped with an MS-r3 or MS-r4 rotor at a frequency of 60 Hz,

a melting point of 25-70° C. and preferably 25-55° C.

Thus, the fatty phase may comprise polymers chosen from polyisobutylenes with a relative molar mass of greater than 150,000, such as, for example, polyisobutylenes with respective relative molar masses Mv of 200,000, 400,000 and 1,110,000, sold under the respective trade names “Oppanol B 30 SF”, “Oppanol B 50 SF” and “Oppanol B 100” by the company BASF, the polyisobutylenes with relative molar masses Mv of between 900,000 and 2,200,000 sold under the trade name “Vistanex MM” by the company Exxon, and mixtures thereof.

The compositions of the invention may also comprise at least one alkyl, alkoxy or phenyl dimethicone such as, for example, the product sold under the name “Abil Wax 2440” by the company Goldschmidt.

The compositions according to the invention may also comprise at least one silicone resin comprising a combination of units R₃SiO_{1/2}, R₂SiO_{2/2}, RSiO_{3/2} and SiO_{4/2}, in which R denotes an alkyl radical containing from 1 to 6 carbon atoms.

The fatty phase may also contain at least one liposoluble dye. This liposoluble dye is, for example, Sudan red, DC Red 17, DC Green 6, β-carotene, soybean oil, Sudan brown, DC Yellow 11, DC Violet 2, DC Orange 5, or quinoline yellow, and mixtures thereof. It can represent from 0.01 to 20% of the total weight of the composition, and better still from 0.1% to 6%.

The fatty phase can represent from 0.1% to 99% and preferably from 0.1% to 80% by weight relative to the total weight of the final composition.

The support of the composition A according to the invention may comprise at least one organic solvent phase, in particular when the composition is a nail varnish.

As organic solvent that may be used in the invention, mention may be made of:

ketones that are liquid at room temperature, such as methyl ethyl ketone, methyl isobutyl ketone, diisobutyl ketone, isophorone, cyclohexanone or acetone;

alcohols that are liquid at room temperature, such as ethanol, isopropanol, diacetone alcohol, 2-butoxyethanol or cyclohexanol;

glycols that are liquid at room temperature, such as ethylene glycol, propylene glycol, pentylene glycol or glycerol;

propylene glycol ethers that are liquid at room temperature, such as propylene glycol monomethyl ether, propylene glycol monomethyl ether acetate or dipropylene glycol mono-n-butyl ether;

short-chain esters (containing from 3 to 8 carbon atoms in total), such as ethyl acetate, methyl acetate, propyl acetate, n-butyl acetate or isopentyl acetate;

ethers that are liquid at room temperature, such as diethyl ether, dimethyl ether or dichlorodiethyl ether;

alkanes that are liquid at room temperature, such as decane, heptane, dodecane or cyclohexane;

cycloaromatic compounds that are liquid at room temperature, such as toluene and xylene;

aldehydes that are liquid at room temperature, such as benzaldehyde or acetaldehyde,

mixtures thereof.

The organic solvent may be present in the composition of the invention in a content ranging from 0.01% to 99% by weight and preferably from 50% to 99% by weight, for use as a nail varnish, relative to the total weight of the composition.

The support of the composition A according to the invention may comprise an aqueous phase. This aqueous phase may comprise water, a floral water such as cornflower water and/or a mineral water such as eau de Vittel, eau de Lucas or eau de La Roche Posay and/or a spring water.

The aqueous phase may also comprise solvents other than water, such as, for example, primary alcohols such as ethanol and isopropanol, glycols such as propylene glycol, butylene glycol, dipropylene glycol or diethylene glycol, glycol ethers such as mono-, di- or tripropylene glycol (C₁-C₄)alkyl ethers, and mono-, di- or triethylene glycol, and mixtures thereof.

The aqueous phase may also comprise water-soluble colorants chosen from the colorants that are common in the field under consideration, such as the disodium salt of ponceau, the disodium salt of alizarin green, quinoline yellow, the trisodium salt of amaranth, the disodium salt of tartrazine, the monosodium salt of rhodamine, the disodium salt of fuchsin, or xanthophyll.

The aqueous phase may also comprise any compound that is compatible with an aqueous phase, such as gelling agents, film-forming polymers, thickeners, surfactants, preserving agents or pigment dispersions, and mixtures thereof.

When the composition according to the invention is a nail varnish, the aqueous phase may consist essentially of water or of an aqueous-alcoholic mixture especially comprising C₁-C₅ monoalcohols or C₂-C₈ glycols.

Preferably, the aqueous phase is present in the compositions of the invention in a content ranging from 0.1% to 99% by weight relative to the total weight of the composition.

The support of the composition A may also comprise at least one amphiphilic compound, i.e. a compound comprising both a lipophilic portion (apolar portion) and a hydrophilic portion (polar portion), which may be adsorbed onto a surface or an interface. Such compounds are, for example, emulsifiers and coemulsifiers.

The emulsifiers and coemulsifiers used in the composition A, when it is in emulsion form, are chosen from those conventionally used in the fields of cosmetics and dermatology. The emulsifier and the coemulsifier may be present in the composition in a proportion preferably ranging from 0.3% to 30% by weight and better still from 0.5% to 20% by weight, relative to the total weight of the composition.

O/W surfactants that may especially be mentioned include (CTFA): cetearylglucoside, PEG-40 stearate, sorbitan tristearate, sorbitan stearate, polysorbate 60, sorbitan stearate/sucrose cocoate mixture, glyceryl stearate/PEG-100 stearate mixture, PEG-400, glyceryl stearate, PEG-6/PEG-32/glycol stearate mixture. W/O surfactants that may especially be mentioned include the polyglyceryl-4 isostearate/cetyldimethicone copolyol/hexyl laurate mixture and the mineral oil/petrolatum/ozokerite/-glyceryl oleate/lanolin alcohol mixture.

The support of the composition A according to the invention may additionally comprise at least one pulverulent compound. The pulverulent compounds may be chosen from the pigments and/or nacres and/or fillers and/or mixtures thereof usually used in cosmetic compositions.

Advantageously, the pulverulent compounds are present in a content ranging from 0.1% to 99.9% and preferably from 60% to 99.9% and more preferably from 80% to 99.9% by weight, relative to the total weight of the composition.

The pigments may be white or colored, and mineral and/or organic. Among the mineral pigments that may be mentioned are titanium dioxide, optionally surface-treated, zirconium oxide or cerium oxide, also iron oxide or chromium oxide, manganese violet, ultramarine blue, chromium hydrate and ferric blue. Among the organic pigments that may be mentioned are carbon black, pigments of D & C type, and lakes based on cochineal carmine or on barium, strontium, calcium or aluminum.

The nacreous pigments may be chosen from white nacreous pigments such as mica coated with titanium or with bismuth oxychloride, colored nacreous pigments such as titanium mica with iron oxides, titanium mica with, especially, ferric blue or chromium oxide, titanium mica with an organic pigment of the abovementioned type, and also nacreous pigments based on bismuth oxychloride.

The fillers may be mineral or organic, and lamellar or spherical. Mention may be made of talc, mica, silica, kaolin, Nylon powders (Orgasol from Atochem or Nylon 12), poly-β-alanine powders and polyethylene powders, Teflon, bismuth oxychloride, lauroyllysine, starch, boron nitride, tetrafluoroethylene polymer powders, hollow microspheres such as Expancel (Nobel Industrie), Polytrap (Dow Corning) and silicone resin microbeads (for example Tospearls from Toshiba), precipitated calcium carbonate, magnesium carbonate, magnesium hydrocarbonate, hydroxyapatite, hollow silica microspheres (Silica Beads from Maprecos), glass or ceramic microcapsules, metal soaps derived from organic carboxylic acids containing from 8 to 22 carbon atoms and preferably from 12 to 18 carbon atoms, for example zinc, magnesium or lithium stearate, zinc laurate or magnesium myristate.

In a known manner, the support for composition A may also contain at least one adjuvant that is common in cosmetics or dermatology, such as hydrophilic or lipophilic gelling agents, thickeners, hydrophilic or lipophilic additives, preserving agents, antioxidants, solvents, fragrances, sunscreens, active agents, odor absorbers and dyestuffs. The amounts of these various adjuvants are those that are conventionally used in these fields, and, for example, from 0.01% to 10% relative to the total weight of the composition. Depending on their nature, these adjuvants may be introduced into the fatty phase, into the organic phase, into the aqueous phase and/or into organized systems.

As hydrophilic gelling agents that may be used in the invention, mention may be made of carboxy-vinyl polymers (carbomer), acrylic copolymers such as acrylate/alkylacrylate copolymers, polyacrylamides, polysaccharides such as hydroxypropylcellulose, natural gums and clays, for instance laponites, and associative thickeners, for instance associative polyurethanes, and lipophilic gelling agents that may be mentioned include modified clays, for instance bentones, and metal salts of fatty acids, for instance aluminum stearates.

As cosmetic, dermatological or hygiene active agents that may be used in the composition of the invention, mention may be made of moisturizers, antioxidants, vitamins, essential fatty acids and sphingolipids. Mention may be made, for example, of polyols such as glycerol, glycols and sugar derivatives, enzymes, vitamins such as vitamin C (ascorbic acid), vitamin A (retinol), vitamin D, vitamin E (tocopherol), vitamin K and derivatives of these vitamins such as the esters, ceramides, depigmenting agents such as kojic acid and caffeic acid, β-hydroxy acids such as salicylic acid and its derivatives, α-hydroxy acids such as lactic acid and glycolic acid, moisturizers such as protein hydrolyzates, softeners such as allantoin, and mixtures thereof.

These active agents may be present in a proportion of from 0.001 to 20% by weight relative to the total weight of the composition.

Needless to say, a person skilled in the art will take care to select this or these optional additional compound(s), and/or the amount thereof, such that the advantageous properties of the composition according to the invention are not, or are not substantially, adversely affected by the envisioned addition.

Composition A may be in any presentation form for topical use normally used and may especially be in the form of an oily suspension or dispersion, a solution containing a volatile solvent (varnish), emulsions obtained by dispersing a fatty phase in an aqueous phase (O/W) or conversely (W/O), triple emulsions (W/O/W or O/W/O) or vesicular dispersions of ionic and/or nonionic type, an anhydrous gel, or free, compact or pressed powders. These compositions are prepared according to the usual methods. According to one preferred embodiment of the invention, composition A is in the form of a W/O, O/W or multiple emulsion, or in the form of a gel and is a makeup base composition.

Composition B according to the invention is a standard cosmetic composition comprising at least one cosmetically acceptable support as defined above for composition A. It may optionally also comprise an adhesive material as defined above.

The compositions B according to the invention are preferably makeup compositions for the skin, semi-mucous membranes, mucous membranes and/or the integuments, and they are then, for example, in the form of a foundation, a rouge, an eyeshadow, a concealer, a body makeup product, a powder, a lipstick, a mascara, an eyeliner or a nail varnish. They may be in any of the standard forms of makeup compositions, such as sticks, tubes, soft pastes, creams, fluids, and free, compact or pressed powders.

Compositions A and B are prepared according to the usual methods known to those skilled in the art. They are in the form of a makeup kit or care kit.

According to the process of the present invention, one of the two compositions A or B is applied to the skin and/or mucous membranes and/or semimucous membranes and/or the integuments. Next, the second composition is applied over the first. The makeup product obtained on the skin by this process has noteworthy staying-power and transfer-resistance properties.

The invention is illustrated in greater detail by the examples that follow.

In the examples which follow the quantities are given as percentages by weight relative to the total weight of the composition.

### EXAMPLE 1:

The Applicant prepared the cosmetic base A below in the form of a W/O emulsion:

Composition A:

Phase A:

| | |
|---|---|
| sorbitan isostearate | 4.2% |
| preserving agent | 0.2% |

Phase B1:

| | |
|---|---|
| hydrogenated isobutylene | 12% |

Phase B2:

| | |
|---|---|
| polyisobutylene of molecular mass | |
| 40,000 sold under the trade name | |
| “Oppanol B 10” by the company BASF | 4% |
| isododecane | 10% |

Phase C:

| | |
|---|---|
| water | 70.7% |
| magnesium sulfate | 0.7% |
| preserving agent | 0.2% |

This emulsion was prepared according to the standard methods for preparing W/O emulsions. Phase A was first prepared by mixing. Phase B1 was then added with stirring while heating. Phase B2 is added after predissolving the polyisobutylene in the isododecane while heating. This results in an oily phase, which is heated for 5 minutes at 80° C. This mixture is cooled to 20° C. All the compounds of phase C are mixed together and then heated until the preserving agent has dissolved. Next, phase C is cooled to 20° C. and then added slowly to the mixture A+B1+B2.

The Applicant then prepared the composition B below. This composition is a face powder.

Composition B1:

| | |
|---|---|
| acetyl tributyl citrate | 8% |
| iron oxide (red) | 5% |
| talc | 87% |

Composition A is applied, for example to a person's face. Composition B is then applied. The product obtained has noteworthy staying power and shows very little transfer.

### EXAMPLE 2:

The Applicant prepared the cosmetic base A below in the form of a solution:

Composition A:

| | |
|---|---|
| polyisobutylene of molecular mass | 10% |
| 40,000 sold under the trade name “Oppanol | |
| B 10” by the company BASF | |
| heptane | 90% |

The Applicant then prepared composition B below, which is a nail varnish.

Composition B:

| | |
|---|---|
| nitrocellulose | 19% |
| N-ethyl-o,p-toluenesulfonamide | 6% |
| acetyl tributyl citrate | 6% |
| pigments | 1% |
| hectorite | 1.2% |
| isopropyl alcohol | 8% |
| ethyl acetate, butyl acetate | qs 100% |

The composition was prepared by simple mixing of the various compounds.

Composition A (base) is applied to the nails or false nails of an individual. Composition B (varnish) is then applied over composition A. The product obtained has noteworthy staying power.

1. A makeup or care kit comprising at least two cosmetic compositions A and B, characterized in that:
composition A comprises at least one adhesive material that satisfies the following conditions:
G′(2 Hz, 35° C.)≧10³ Pa, and
G′(35° C.)≦10⁸ Pa, preferably G′(35° C.)≦10⁷ Pa,
G′(2×10⁻² Hz, 35° C.)≦3×10⁵ Pa, and
in which:
G′(2 Hz, 35° C.) is the elastic shear modulus of said adhesive material, measured at a frequency of 2 Hz and at a temperature of 35° C.,
G′(35° C.) is the elastic shear modulus of said adhesive material, measured at a temperature of 35° C., for any frequency between 2×10⁻² and 2 Hz,
G′(2×10⁻² Hz, 35° C.) is the elastic shear modulus of said adhesive material, measured at a frequency of 2×10⁻² Hz and at a temperature of 35° C.

2. The kit as claimed in claim 1, such that the adhesive material also satisfies the following condition:
G″/G′(0.2 Hz, 35° C.)≧0.35,
in which
G″(0.2 Hz, 35° C.) is the viscous shear modulus of said adhesive material, measured at a frequency of 0.2 Hz and at a temperature of 35° C.,
G′(0.2 Hz, 35° C.) is the elastic shear modulus of said adhesive material, measured at a frequency of 0.2 Hz and at a temperature of 35° C.

3. The kit as claimed in claim 1 or 2, such that:
G′(2 Hz, 35° C.)≧5×10³ Pa and better still G′(2 Hz, 35° C.)≧10⁴ Pa.

4. The kit as claimed in any one of claims 1 to 3, such that:
G′(2×10⁻² Hz, 35° C.)≦5×10⁴ Pa.

5. The kit as claimed in any one of the preceding claims, such that:
G′(2 Hz, 35° C.)≧10⁴ Pa, and
G′(35° C.)≦10⁸ Pa, preferably G′(35° C.)≦10⁷ Pa,
G′(2×10−2 Hz, 35° C.)≦5×10⁴ Pa, and
G″/G′(0.2 Hz, 35° C.)≧0.35.

6. The kit as claimed in any one of the preceding claims, such that the adhesive materials are chosen from block and random copolymers comprising at least one monomer or a combination of monomers whose resultant polymer has a glass transition temperature of less than room temperature (25° C.), these monomers or combinations of monomers possibly being chosen from butadiene, ethylene, propylene, isoprene, isobutylene and a silicone, and blends thereof.

7. The kit as claimed in any one of the preceding claims, such that the adhesive materials are chosen from:
polyurethanes,
acrylic polymers,
silicones,
butyl rubbers, especially from polyisobutylenes,
ethylene/vinyl acetate polymers,
polyamides optionally modified with fatty chains,
natural rubbers,
and blends thereof.

8. The kit as claimed in any one of the preceding claims, such that the adhesive materials are chosen from polyisobutylenes with a relative molar mass Mv of greater than or equal to 10,000 and less than or equal to 150,000 and more preferably greater than or equal to 18,000 and less than or equal to 150,000.

9. The kit as claimed in any one of the preceding claims, such that the adhesive materials are present in a content ranging from 0.1% to 99%, preferably from 0.1% to 30% and more preferably from 0.1% to 10% by weight, relative to the total weight of the composition A.

10. The kit as claimed in any one of the preceding claims, such that the composition A comprises a cosmetically acceptable support.

11. The kit as claimed in any one of the preceding claims, such that composition A is in the form of a W/O, O/W or multiple emulsion, or in the form of a gel.

12. The kit as claimed in any one of the preceding claims, such that composition B comprises a cosmetically acceptable support.

13. The kit as claimed in any one of the preceding claims, such that composition B is in the form of a makeup composition for the skin, semimucous membranes, mucous membranes and/or the integuments.

14. The kit as claimed in the preceding claim, such that composition B is in the form of a foundation, a rouge, an eyeshadow, a concealer, a body makeup product, a powder, a lipstick, a mascara, an eyeliner or a nail varnish.

15. A process for improving the staying-power and transfer-resistance properties of a cosmetic composition A or B, which consists in applying to the skin and/or mucous membranes and/or semimucous membranes and/or integuments, successively and in any order, the two compositions A and B, at least one of the two compositions A and B comprising at least one adhesive material that satisfies the following conditions:
G′(2 Hz, 35° C.)≧10³ Pa, and
G′(35° C.)≦10⁸ Pa, preferably G′(35° C.)≦10⁷ Pa,
G′(2×10⁻² Hz, 35° C.)≦3×10⁵ Pa, and
in which:
G′(2 Hz, 35° C.) is the elastic shear modulus of said adhesive material, measured at a frequency of 2 Hz and at a temperature of 35° C.,
G′(35° C.) is the elastic shear modulus of said adhesive material, measured at a temperature of 35° C., for any frequency between 2×10⁻² and 2 Hz,
G′(2×10⁻² Hz, 35° C.) is the elastic shear modulus of said adhesive material, measured at a frequency of 2×10⁻² Hz and at a temperature of 35° C.
